(19) United States
(12) Patent Application Publication
(10) Pub. No.: US 2003/0055023 A1
(43) Pub. Date: Mar. 20, 2003

(54) Formulations containing etomidate and a sulfoalkyl ether cyclodextrin

(76) Inventors: Roger A. Rajewski, Lawrence, KS (US); Michelle P. McIntosh, Lawrence, KS (US); Diane O. Thompson, Overland Park, KS (US); Gerold L. Mosher, Kansas City, MO (US)

Correspondence Address: INNOVAR, LLC, P O BOX 250647, PLANO, TX, 75025, US

(21) Appl. No.: 10/102,049

(22) Filed: Mar. 19, 2002

Related U.S. Application Data
(60) Provisional application No. 60/277,305, filed on Mar. 20, 2001.

(51) Int. Cl. A61K 31/724

(52) U.S. Cl. 514/58

(57) ABSTRACT

An injectable formulation of a sedative hypnotic drug, such as the anesthetic drug etomidate, that is pharmaceutically stable, demonstrates a reduced incidence of pain upon injection, and is bioequivalent with currently approved formulations. The formulation of the present invention employs a sulfoalkyl ether cyclodextrin solubilizing and complexing excipient, such as CAPTISOL® cyclodextrin (sulfobutyl ether β-cyclodextrin) to form a true aqueous solution. This formulation minimizes the allergic response and microbial contamination issues typically associated with parenteral emulsion formulations. The present formulation also reduces pain on injection as compared to the known organic solvent based formulations containing etomidate. The liquid formulation can be sterile filtered unlike emulsion-type formulations of sedative hypnotics. The liquid formulation can be lyophilized or otherwise dried to yield a solid formulation.

### FIELD OF THE INVENTION

The present invention relates to anesthetic liquid formulations and in particular to a parenteral formulation containing a sedative hypnotic agent, such as etomidate, and a sulfoalkyl ether cyclodextrin and to the use of this formulation.

### BACKGROUND OF THE INVENTION

Etomidate ((R)-(+)ethyl 1-(1-phenylethyl)-1H-imidazole-5-carboxylate) and is an injectable potent, short-acting, non-barbiturate sedative-hypnotic agent for use in the induction and maintenance of anesthesia or sedation. Intravenous injection of a therapeutic dose of etomidate rapidly induces anesthesia usually within 10 or 40 seconds, respectively, from the start of injection. As with other rapidly acting intravenous anesthetic agents, the half-time of the blood-brain equilibrium is approximately 1 to 3 minutes and this accounts for the rapid induction of anesthesia.

Etomidate has been reported to hydrolyze in solution to its free acid (R)-(+) 1-(phenylethyl)-1H-imidazole-5-carboxylic acid. Under conditions of reflux in acid systems, the hydrolysis is accelerated. Almost total degradation to other products occurs in strong base reflux. In neutral reflux, as well as under exposure to low heat or ambient light, very little degradation occurs.

Etomidate is a poorly water-soluble drug having an aqueous solubility of about 0.0045 mg/100 mL (Analytical Profiles of Drug Substances. vol. 12 p. 201 (1983)). Etomidate has been formulated in propylene glycol, lipid emulsions, polyethylene glycol, and phosphate buffers. The original formulation of etomidate was in a phosphate buffer with a pH of 3.3 and an osmolality of 270 mOsm/kg. The phosphate buffer solution, however, was unstable for long-term storage. As a consequence etomidate was re-formulated in a 35% propylene glycol solution having an osmolality of 4900 mOsm/kg. Because it was recognized early on that etomidate solved in propylene glycol can undergo transesterification, the search for a new formulation has been in process for quite some time (U.S. Pat. No. 4,289,783 to Mesens).

Commercially available formulations of etomidate are clear, colorless solutions containing etomidate, propylene glycol and water. The currently marketed AMIDATE® (Abbott Laboratories, Abbott Park, Ill.), HYPNOMIDATE® (Janssen Pharmaceutica Ltd., South Africa), and Etomidate Injection formulations contain 2 mg/mL of etomidate dissolved in 35% vol. propylene glycol and water. Other formulations include etomidate in lipid emulsion, Etomidate-Lipuro®, (10% medium-chain triglyceride and 10% soybean emulsion, B. Braun, Melsungen, Germany) and an experimental formulation of etomidate in a solution containing hydroxypropyl-β-cyclodextrin (HP-β-CD) (Janssen, Neuss, Germany).

Injection of a therapeutic amount of etomidate formulated in 35% propylene glycol often results in hemolysis, myoclonic reactions, transient venous pain, pain on injection, injury to vascular endothelium, venous sequelae, thrombosis, and/or thrombophlebitis (A. W. Doenicke et al. in Br. J. Anesth. (1999) 83 (3):464-6). It has been suggested that the extremely high osmolality (4900-4965 mOsm/kg) of the commercially available propylene glycol-containing preparations causes the severe pain on injection and subsequent thrombophlebitis and disrupting erythrocytes (M. Mayer et. al in Anesth. 1992 vol. 77 pp. A414). The incidence of pain on injection appears to be less frequently noted when larger, more proximal arm veins are employed and it appears to be more frequently noted when smaller, more distal, hand or wrist veins are employed (Bedford Laboratories, Etomidate Injection Product Insert, October 1998). Moreover, the infusion of propylene glycol containing solutions has been associated with hematuria in rats and dogs (Fort, F.L. et al., “Hemolysis Study of Aqueous Polyethylene Glycol 400, Propylene Glycol and Ethanol Combinations In Vivo and In Vitro”, J. Parenter. Sci. and Tech. (1984) Vol. 38 No. 2). Acute toxicosis was reported to have been likely caused by propylene glycol in another study (Moon, P. F. “Acute Toxicosis in Two Dogs Associated with Etomidate-Propylene Glycol Infusion”, Laboratory Animal Science. (1994) Vol. 44 No. 6 pp. 590-4). Propylene glycol is known in the art to be associated with systemic toxic effects (E. Napke et al. Excipients and Additives: Hidden Hazards in Drug Products and in Product Substitution, Vet. Hum Toxicol 32 (3); (1990) citing Martin, G and Finberg, L. Propylene Glycol: a potentially toxic vehicle in liquid dosage form. J. Pediatr 77: 877-878, (1970)).Among the effects observed include: central nervous system effects such as: signs of stupor, azotemia, increased anion gap, and lactic acidosis (Id. citing Cate, J. C., and R. Hedrick. 1980 “Propylene glycol intoxication and lactic acidosis”, N. Eng. J. Med. 303:1237; Martin, G., and L. Finberg. 1970. “Propylene glycol: A potentially toxic vehicle in liquid dosage forms”, J. Pediatr. 77:877-878.; Arulanatham, K., and M. Genel. 1978. “Central Nervous system toxicity associated with ingestion of propylene glycol”, J. Pediatr. 93:515-516.). Propylene glycol has been associated with a protracted seizure unresponsive to anticonvulsant medication (E. Napke et al. Excipients and Additives: Hidden Hazards in Drug Products and in Product Substitution, Vet. Hum Toxicol 32 (3); (1990) citing Arulanantham, K. et al. Central nervous system toxicity associated with ingestion of propylene glycol. J. Pediatr. 93:515-516, (1978)). In this case, the patient's electroencephalogram, grossly abnormal during the seizure, reverted to normal when the exposure to propylene glycol was stopped.

Several patents and scientific publications disclose compositions containing etomidate with improved stability or solubility and/or decreased side effects. With respect to hemolysis, a lipid emulsion is reportedly superior to propylene glycol as a solvent for etomidate (A. Doenicke et al. in Br. J. Anaesth. (1997) vol. 79 pp. 386-8). A 40% ethanol in saline solution caused hematuria in rats, but a 30% ethanol in saline solution did not (F. L. Fort et al. in J. Parenter Sci Technol. (1984) vol. 38 (2) pp. 82-7). U.S. Pat. No. 4,289,783 to Mesens discloses an improved etomidate-containing infusion liquid containing ethanol. U.S. Pat. Nos. 5,024,998 and 4,983,586 to Bodor disclose a method for decreasing the incidence of precipitation of lipophilic and/or water-labile drugs including etomidate by administration in an aqueous solution containing hydroxypropyl-β-cyclodextrin. U.S. Pat. No. 5,646,131 to Badwan, et al. discloses a method for enhancing the solubility of a drug including etomidate, which is insoluble or sparingly soluble in water with cyclodextrin. None of these references describe, disclose or suggest the formulation of the present invention.

Several other methods have been tested in attempts to reduce the above-described side-effects associated with the current formulations of etomidate. These include dilution of the preparation with sodium chloride; injection of the etomidate formulation into a running infusion; premedication of the patient with opioids, benzodiazepines, or local anesthetics; and replacement of the solvent with phosphate buffer, Cremophor EL®, polyethylene glycol, ethanol, fat emulsion, or propylene glycol (A. Doenicke et al. “A comparison of Two Formulations for Etomidate, 2-Hydroxypropyl-β-cyclodextrin (HPCD) and Propylene Glycol”, Anesth. Analg 1994:79:933-9 citing R. J. Stockham et. al., “Fentanyl pretreatment modifies anaesthetic induction of etomidate”, Anaesth. Intensive care (1988) vol. 16 pp. 171-6; A. Hodcroft et. al., “Effect of dose and premedication on induction complications with etomidate”, Br. J. Anaesth. (1976) vol. 48 pp. 199-205; V. Shuermans et. al., “Multinational evaluation of etomidate for anesthesia induction”; Anaesthesist (1978) vol. 27 pp. 52-9; A. Hodcroft et. al., “Effect of dose and premedication on induction complications with etomidate”, Br. J. Anaesth. (1976) vol. 48 pp. 199-205; M. Zacharias et. al., “Venous sequelae following etomidate”, Br. J. Anesth. (1979) vol. 51 pp. 779-83; B. Kay, “A clinical assessment of the use of etomidate in children”; Br. J. Anesth. (1976) vol. 48 pp. 207-11; J. G. B. Hendry et. al., “Etomidate in a new solvent”, Anaesth. (1977) vol. 32 pp. 996-9; R. Ganta et. al., “Pain on injection and venous sequelae following two forms of etomidate”, Eur. J. Anaesth. (1989) vol. 6 pp. 431-4; L. Gran et. al., “Eine Lösung zur schmerzfreign Injektion”, Anaesthesist (1983) vol. 32 pp. 475-7; and A. Doenicke et al., “Etomidat mit einem neuen Losungsvermittler.Klinish-experimentelle Untersuchungen zur Venenvertr\overline{a}glichkeit und Bioverfügbarkei”, Anaesthesist (1983) vol. 39 pp. 475-80). However, none of the above described methods have had an appreciable effect on the reduction of side effects associated with the marketed formulations. As such, there remains a continuing need in the art for a safer formulation of etomidate which does not have the potential to cause the above described side-effects.

In a comparison study of formulations of etomidate in propylene glycol and 2-hydroxypropyl-β-cyclodextrin (HPCD), researchers concluded that HPCD may be superior to propylene glycol as a solvent for etomidate, HPCD being associated with less pain, less thrombophlebitis, and apparently no hemolysis at the levels of administration tested, however, concerns exist as to impairment of renal function in hydroxypropyl-β-cyclodextrin preparations (A. Doenicke et al. in Anesth. Analg (1994) vol. 79 pp. 933-9; A. E. Nebauer et al. Anesthesiology (1992), Sep., 77(3a), A409). The HPCD-containing formulation (comprising HPCD at 30 mg/ml and etomidate at 2 mg/ml) reportedly did not alter the pharmacodynamics or pharmacokinetics of etomidate in a clinically important manner. The researchers also recognized the continuing need for a formulation containing an inert solvent for etomidate so as not to cause side effects, attributed to the excipients. (. A. Doenicke et al. in Anesth. Analg. (1994) vol. 79 pp. 933-9 citing Napke E, Stevens DGH. Excipients and additives: hidden hazards in drug products and in product substitution. Vet. Hum. Toxicol. (1990) vol.32 pp. 352-6).

Clearly, an etomidate formulation, which exhibits a marked decline in the above-described side effects or which provides other benefits, would be preferred.

Numerous studies have reported on the success of formulations containing cyclodextrins and cyclodextrin derivatives to reduce tissue damage and pain following intramuscular injection (T. Irie et al. J. Pharmacobio-Dyn. (1983) 6(10):790-2; K. Masuda et al. in Yakugaku Zasshi (1984) 104(10):1075-82; A. Yoshida et al. in Chem. Pharm. Bull. (1990) 38(1):176-9) and intradermal injection (U.S. Pat. No. 5,602,112 to J. Rubinfeld). The intramuscular studies evaluated the tissue irritation after drugs were administered as suspensions in saline, or solubilized as complexes with β-cyclodextrin or 2-hydroxypropyl-β-cyclodextrin in water. The formulations containing the drugs complexed with the cyclodextrins showed reduced visual signs of irritation and tissue damage as compared to the formulations in saline. No assessment was made of pain. These studies show reduced tissue damage from cyclodextrin complexation, but only after 2 days of localized contact. The intradermal studies evaluated the ulcerative effects of several cytotoxic compounds, formulated with or without cyclodextrins, after administration into the skin of rats. Again, no measurement of pain was taken and the irritation evaluation was conducted only after contact times of 1 to 20 days. None of these studies evaluated the effects of cyclodextrin complexation on the pain associated with injection, especially after rapid intravenous administration or a continuous intravenous infusion.

Cyclodextrins are cyclic carbohydrates derived from starch. The unmodified cyclodextrins differ by the number of glucopyranose units joined together in the cylindrical structure. The parent cyclodextrins contain 6, 7, or 8 glucopyranose units and are referred to as α-, β-, and γ-cyclodextrin respectively. Each cyclodextrin subunit has secondary hydroxyl groups at the 2 and 3 positions and a primary hydroxyl group at the 6 position. The cyclodextrins may be pictured as hollow truncated cones with hydrophilic exterior surfaces and hydrophobic interior cavities. In aqueous solutions, these hydrophobic cavities provide a haven for hydrophobic organic compounds that can fit all or part of their structure into these cavities. This process, known as inclusion complexation, may result in increased apparent aqueous solubility and stability for the complexed drug. The complex is stabilized by hydrophobic interactions and does not involve the formation of any covalent bonds.

This dynamic and reversible equilibrium process can generally be described by Equations 1 and 2, where the amount in the complexed form is a function of the concentrations of the drug and cyclodextrin, and the equilibrium or binding constant, K_{b}. When cyclodextrin formulations are administered by injection into the blood stream, the complex rapidly dissociates due to the effects of dilution and non-specific binding of the drug to blood and tissue components.

$$\begin{matrix}Drug + Cyclodextrin {⟷}^{{K}_{b}} Complex & Equation 1 \\ {K}_{b} = \frac{[ Complex ]}{[ Drug ] [ Cyclodextrin ]} & Equation 2\end{matrix}$$

The underivatized parent cyclodextrins are known to interact with and extract cholesterol and other membrane components, particularly upon accumulation in the kidney tubule cells, leading to toxic and sometimes fatal renal effects. Chemical modification of the parent cyclodextrins (usually at the hydroxyls) has resulted in derivatives with improved safety while retaining or improving the complexation ability. Of the numerous derivatized cyclodextrins prepared to date, only two appear to be commercially viable: the 2-hydroxypropyl derivatives (HP-β-CD; neutral cyclodextrins being commercially developed by Janssen and others), and the sulfoalkyl ether derivatives (SAE-CD), such as sulfobutyl ether, (SBE-CD; anionic cyclodextrins being developed by CyDex, Inc.). However, the HP-β-CD still possesses toxicity that the SBE-CD does not.

A sulfobutyl ether derivative (SBE-β-CD), in particular the derivative with an average of about 7 substituents per cyclodextrin molecule, is being commercialized by CyDex, Inc. as CAPTISOL® cyclodextrin. The anionic sulfobutyl ether substituent

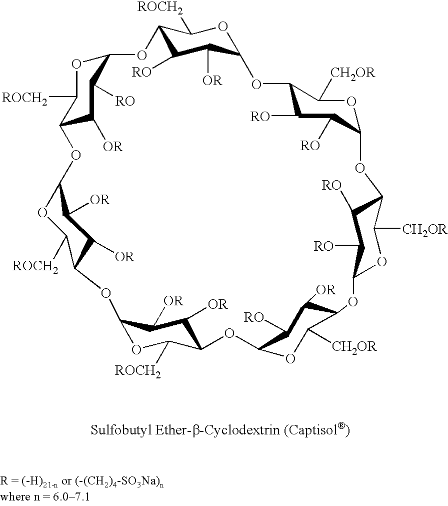

dramatically improves the aqueous solubility of the cyclodextrin. In addition, the presence of the charges decreases the ability of the molecule to complex with cholesterol as compared to the hydroxypropyl derivative. Reversible, non-covalent, complexation of drugs with CAPTISOL® cyclodextrin generally allows for increased solubility and stability of drugs in aqueous solutions. While CAPTISOL® cyclodextrin is a relatively new but known cyclodextrin, its combined use with etomidate in parenteral formulations and its effect upon the behavior of etomidate when administered parenterally has not previously been evaluated.

The safety of cyclodextrins is often compared by way of in-vitro hemolysis studies. As depicted in FIG. 1 (Thompson, D. O., Critical Reviews in Therapeutic Drug Carrier Systems, 14(1), 1-104 (1997)), the hemolytic behavior of the CAPTISOL® cyclodextrin is compared to the same for the parent β-cyclodextrin, the commercially available hydroxypropyl derivatives, ENCAPSIN™ cyclodextrin (degree of substitution˜3-4) and MOLECUSOL® cyclodextrin (degree of substitution˜7-8), and two other sulfobutyl ether derivatives, SBE1-β-CD and SBE4-β-CD. Unlike the other cyclodextrin derivatives, SAE-CD derivatives, in particular those such as the CAPTISOL® cyclodextrin (degree of substitution˜7) and SBE4-β-CD (degree of substitution˜4), show essentially no hemolytic behavior in concentrations typically used to solubilize pharmaceutical formulations. These SAE-CDs exhibit substantially lower membrane damaging potential than the commercially available hydroxypropyl derivatives.

Sulfated cyclodextrin derivatives have also been prepared and their effects on blood clotting time evaluated. Sulfated cyclodextrins were found to interfere significantly with blood clotting time, especially when compared to the sulfoalkyl ether cyclodextrins (Thompson, D. O., Critical Reviews in Therapeutic Drug Carrier Systems, (1997), 14(1), 1-104).

Methylated cyclodextrins have been prepared and their hemolytic effect on human erythrocytes has been evaluated. These cyclodextrins were found to cause moderate to severe hemolysis (Jodal et al., Proc. 4^{th} Int. Symp. Cyclodextrins, (1988), 421-425; Yoshida et al., Int. J. Pharm., (1988), 46(3), 217-222).

Accordingly, of the different cyclodextrins mentioned above, sulfoalkyl ether cyclodextrins possess preferential qualities for use in parenteral formulations.

Thus, in the field of sedative hypnotic therapy, especially etomidate therapy, there remains the need for improved injectable formulations that have a reduced or eliminated incidence of pain upon injection, reduced or eliminated hemolytic potential, enhanced stability, minimal potential for allergic reaction and microbial growth, and/or minimal cardiac side effects caused by the formulation.

### SUMMARY OF THE INVENTION

The present invention seeks to overcome the disadvantages present in known formulations. As such, a sulfoalkyl ether cyclodextrin (SAE-CD)-based parenteral formulations of a sedative hypnotic, such as etomidate, is provided. The present formulation may provide reduced pain on injection as compared to commercially available organic solvent based formulations. In addition, the present formulation is pharmaceutically stable and has a reduced potential for bacterial contamination, allergic reaction to the formulation components, and hyperlipidemia in recipients as compared to lipid emulsion-based formulations of etomidate. Additionally, unlike an HP-β-CD or propylene glycol based formulation, the present liquid formulation does not induce hemolysis when administered by injection. Moreover, the present formulation possesses other physicochemical advantages over the HP-β-CD based formulation and the propylene glycol based formulation.

An SAE-CD-containing formulation can be prepared with sufficient etomidate solubility and stability for a commercial product. The CAPTISOL® cyclodextrin formulation of etomidate can be prepared as a clear aqueous true solution that can be sterile filtered (filter pore size of 0.1 to 0.22 microns) and is stable under a variety of storage conditions. The CAPTISOL® cyclodextrin liquid formulation can also be converted to a solid or powder for reconstitution.

One aspect of the invention provides a liquid formulation comprising a therapeutically effective amount of a sedative hypnotic agent, such as etomidate, and a sulfoalkyl ether cyclodextrin present in an amount sufficient to reduce the pain on injection typically associated with the parenteral administration of an etomidate organic solvent-based formulation. The SAE-CD can be present in less than stoichiometric, stoichiometric or greater than stoichiometric amounts with respect to the amount of etomidate present in the formulation.

Specific embodiments of the invention include those wherein: 1) the etomidate to SAE-CD molar ratio is less than one, about one or greater than one; 2) the SAE-CD is sulfobutyl ether 4-β-CD or sulfobutyl ether 7-β-CD; 3) the SAE-CD is a compound of the formula 1 or a mixture thereof; 4) the liquid formulation further comprises a preservative, an antioxidant, a buffering agent, an acidifying agent, a solubilizing agent, a complexation enhancing agent, saline, an electrolyte, another therapeutic agent, an alkalizing agent, an antimicrobial agent, an antifungal agent or a combination thereof; 5) the SAE-CD is present in an amount sufficient to provide a clear solution; 6) the sedative hypnotic agent is etomidate; and/or 7) the liquid formulation is lyophilized or otherwise dried to form a solid formulation for reconstitution.

The invention also provides a liquid formulation of etomidate that can be sterile filtered, wherein the formulation comprises a liquid carrier, an SAE-CD and etomidate. Another aspect of the invention provides a method of reducing or eliminating the pain on injection typically associated with the parenteral administration of a etomidate-containing liquid comprising the step of administering a liquid formulation comprising a sulfoalkyl ether cyclodextrin and etomidate.

The invention also provides a method of administering a sedative hypnotic agent comprising the step of administering a liquid formulation comprising a sulfoalkyl ether cyclodextrin and a sedative hypnotic agent.

Another aspect of the invention provides a method of inducing hypnosis or a method of inducing or maintaining sedation in a mammal comprising the step of administering a liquid formulation comprising an SAE-CD and a sedative hypnotic agent while reducing the pain on injection typically associated with the administration of sedative hypnotic organic solvent-based formulations.

Specific embodiments of the methods of the invention include those wherein: 1) the sedative hypnotic agent is etomidate; 2) the method further comprises the step of administering the liquid formulation by injection, infusion, or orally; 3) the method further comprises the step of mixing the SAE-CD and etomidate, and optionally one or more other ingredients, in a solution to form the liquid formulation; 4) the liquid formulation comprises an excess, on a molar basis, of SAE-CD; 5) the liquid formulation comprises an excess, on a molar basis, of sedative hypnotic agent; 6) the reduced pain on injection is based upon a comparison to the commercially available AMIDATE® organic solvent based formulation; 7) the liquid formulation provides a heart-rate response similar to that of AMIDATE® organic solvent based formulation; 8) the liquid formulation provides an overall blood pressure response similar to that of AMIDATE® organic solvent based formulation with a decreased rate of change in mean arterial pressure; 9) the liquid formulation provides a electroencephalographic response similar to that of AMIDATE® organic solvent based formulation; 10) the liquid formulation provides pharmacokinetics similar to that of AMIDATE® organic solvent based formulation; 11) the liquid formulation provides pharmacodynamics similar to that of AMIDATE® organic solvent based formulation; 12) the liquid formulation provides equivalent or improved chemical stability of etomidate as compared to AMIDATE® organic solvent based formulation; 13) the liquid formulation can be converted to a solid formulation for reconstitution; and 14) the solid formulation provides equivalent or improved chemical stability of etomidate as compared to AMIDATE® organic solvent based formulation.

The invention also provides methods of preparing a sedative hypnotic-based liquid formulation. A first method comprises the steps of: forming a first aqueous solution comprising SAE-CD; forming a second solution comprising etomidate; and mixing the first and second solutions to form the liquid formulation. A second method is similar to the first step except that the etomidate is added directly to the first solution without formation of the second solution. A third method is similar to the first except that the SAE-CD is added directly to the second solution without formation of the first solution. A fourth method comprises the steps of: adding a solution comprising etomidate to a powdered or particulate SAE-CD. A fifth method comprises the steps of: adding the etomidate directly to the powdered or particulate SAE-CD; and adding a second solution. A sixth method comprises the steps of: creating the liquid formulation by any of the above methods and then isolating a solid material by lyophilization, spray- drying, spray-freeze-drying, antisolvent precipitation, a process utilizing a supercritical or near supercritical fluid, or other methods known to those of ordinary skill in the art to make a powder.

Specific embodiments of the methods of preparing the liquid formulation include those wherein: 1) the method further comprises the step of sterile filtering the formulation through a filtration medium having a pore size of 0.45 microns or smaller; 2) the liquid formulation is sterilized by irradiation or autoclaving; 3) the method further comprises the step of isolating a powder from the solution; 4) the solution is purged with nitrogen or argon or other inert pharmaceutically acceptable gas such that a substantial portion of the oxygen dissolved in the solution is removed.

Another aspect of the invention provides a kit comprising: a first pharmaceutical composition comprising an SAE-CD and a second pharmaceutical composition comprising a sedative hypnotic agent. The first and second formulations can be mixed and formulated as a liquid dosage form prior to administration to a subject. Either one or both of the first and second pharmaceutical compositions can comprise additional pharmaceutical excipients.

Specific embodiments of the kit include those wherein: 1) the first and second pharmaceutical compositions are provided in separate containers or in separate chambers of a container having two or more chambers; 2) the kit further comprises a pharmaceutically acceptable liquid carrier used to suspend and dissolve the first and/or second pharmaceutical compositions; 3) the liquid carrier is included with the first and/or second pharmaceutical composition; 4) the liquid carrier is provided in a container or chamber separate from the first and second pharmaceutical compositions; 5) the first and/or second pharmaceutical composition and/or liquid carrier further comprises a preservative, an antioxidant, a complexation enhancing agent, a buffering agent, an acidifying agent, saline, an electrolyte, another therapeutic agent, an alkalizing agent, an antimicrobial agent, an antifungal agent, a solubility enhancing agent or a combination thereof; 6) the kit is provided chilled; 7) the liquid carrier and/or chamber has been purged with a pharmaceutically acceptable inert gas to remove substantially all of the oxygen dissolved in the liquid carrier; 8) the chambers are substantially free from oxygen. Still another aspect of the invention provides a reconstitutable solid pharmaceutical composition comprising a sedative hypnotic agent, an SAE-CD and optionally at least one other pharmaceutical excipient. When this composition is reconstituted with an aqueous liquid to form a liquid formulation that can be administered by injection, infusion, or orally to a subject. The liquid formulation so formed provides a reduced pain on injection as compared to organic solvent-based formulations containing a sedative hypnotic.

Specific embodiments of the reconstitutable solid pharmaceutical composition includes those wherein: 1) the composition comprises an admixture of a solid SAE-CD and sedative hypnotic-containing solid comprising a sedative hypnotic and optionally at least one solid pharmaceutical excipient, such that a major portion of the sedative hypnotic is not complexed with the SAE-CD prior to reconstitution; and/or 2) the composition comprises a solid mixture of an SAE-CD and a sedative hypnotic, wherein a major portion of the sedative hypnotic is complexed with the SAE-CD prior to reconstitution.

Yet another aspect of the invention provides a pharmaceutical kit comprising a first container containing a liquid vehicle and a second container containing a reconstitutable solid pharmaceutical composition comprising an SAE-CD and a sedative hypnotic.

Specific embodiments of this kit include those wherein: 1) the liquid vehicle comprises an aqueous liquid carrier; 2) the composition comprises an admixture of a solid SAE-CD and sedative hypnotic-containing solid comprising a sedative hypnotic and optionally at least one solid pharmaceutical excipient, such that a major portion of the sedative hypnotic is not complexed with the SAE-CD prior to reconstitution; and/or 3) the composition comprises a solid mixture of an SAE-CD and a sedative hypnotic, wherein a major portion of the sedative hypnotic is complexed with the SAE-CD prior to reconstitution.

These and other aspects of this invention will be apparent upon reference to the following detailed description and attached figures. To that end, certain patents and other documents are cited herein to more specifically set forth various aspects of this invention, each of which are hereby incorporated by reference in their entirety.

### BRIEF DESCRIPTION OF THE FIGURES

The following drawings are given by way of illustration only, and thus are not intended to limit the scope of the present invention.

FIG. 1 depicts a prior art graph showing the hemolytic potential for various different cyclodextrin derivatives.

FIG. 2 depicts a graph of the osmolality versus concentration of SBE-CD in an aqueous solution.

FIG. 3 depicts a phase solubility diagram for the binding of etomidate to SBE7-β-CD.

FIG. 4 depicts a plasma versus time blood level profile of etomidate following iv and subcutaneous administration of CAPTISOL® cyclodextrin formulations of etomidate.

### DETAILED DESCRIPTION OF THE INVENTION

A formulation comprising an SAE-CD and etomidate provides unexpected advantages over other formulations comprising etomidate and another cyclodextrin derivative or organic solvent. The presently claimed formulation overcomes many of the undesired properties of other known formulations while at the same time providing a formulation that is bioequivalent with a currently approved propylene glycol based formulation. As used herein, the term etomidate refers to any salt form, salt free form, optically pure, racemic or optically enriched form.

The present invention provides SAE-CD based parenteral formulations of etomidate, wherein the SAE-CD is a compound of the Formula 1 or combinations thereof:

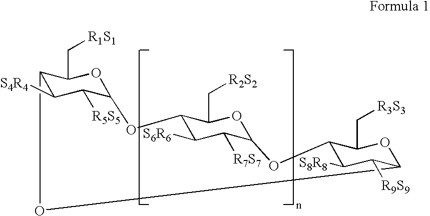

wherein:

n is 4, 5 or 6;

R₁, R₂, R₃, R₄, R₅, R₆, R₇, R₈ and R₉ are each, independently, —O— or a-O—(C₂-C₆ alkylene)-SO₃⁻ group, wherein at least one of R₁ and R₂ is independently a —O—(C₂-C₆ alkylene)-SO₃⁻ group, preferably a —O—(CH₂)ₘSO₃⁻ group, wherein m is 2 to 6, preferably 2 to 4, (e.g. —OCH₂CH₂CH₂SO₃⁻ or —OCH₂CH₂CH₂CH₂SO₃⁻); and

S₁, S₂, S₃, S₄, S₅, S₆, S₇, S₈ and S₉ are each, independently, a pharmaceutically acceptable cation which includes, for example, H⁺, alkali metals (e.g. Li⁺, Na⁺, K⁺), alkaline earth metals (e.g., Ca⁺², Mg⁺²), ammonium ions and amine cations such as the cations of (C₁-C₆)-alkylamines, piperidine, pyrazine, (C₁-C₆)-alkanolamine and (C₄-C₈)-cycloalkanolamine.

The SAE-CD used in the liquid or solid formulations are described in U.S. Pat. Nos. 5,376,645 and 5,134,127 to Stella et al, the entire disclosures of which are hereby incorporated by reference. The preparation process may comprise dissolving the cyclodextrin in aqueous base at an appropriate temperature, e.g., 70° to 80° C., at the highest concentration possible. For example, to prepare the cyclodextrin derivatives herein, an amount of an appropriate alkyl sultone, corresponding to the number of moles of primary CD hydroxyl group present, is added with vigorous stirring to ensure maximal contact of the heterogeneous phase. According to one embodiment, the SAE-CD is SBE-7-β-CD (CAPTISOL® cyclodextrin) or SBE-4-β-CD.

The terms “alkylene” and “alkyl,” as used herein (e.g., in the -0-(C₂-C₆-alkylene)SO₃⁻ group or in the alkylamines), include linear, cyclic, and branched, saturated and unsaturated (i.e., containing one double bond) divalent alkylene groups and monovalent alkyl groups, respectively. The term “alkanol” in this text likewise includes both linear, cyclic and branched, saturated and unsaturated alkyl components of the alkanol groups, in which the hydroxyl groups may be situated at any position on the alkyl moiety. The term “cycloalkanol” includes unsubstituted or substituted (e.g., by methyl or ethyl) cyclic alcohols.

The present invention provides compositions containing a mixture of cyclodextrin derivatives, having the structure set out in formula (1), where the composition overall contains on the average at least 1 and up to 3n+6 alkylsulfonic acid moieties per cyclodextrin molecule. The present invention also provides compositions containing a single type of cyclodextrin derivative, or at least 50% of a single type of cyclodextrin derivative.

The cyclodextrin derivatives of the present invention are obtained as purified compositions, i.e., compositions containing at least 95 wt. % of cyclodextrin derivative(s). In a preferred embodiment, purified compositions containing at least 98 wt. % cyclodextrin derivative(s) are obtained.

In some of the compositions of the invention unreacted cyclodextrin has been substantially removed, with the remaining impurities (i.e., <5 wt. % of composition) being inconsequential to the performance of the cyclodextrin derivative-containing composition.

Exemplary SAE-CD derivatives include SBE4-β-CD, SBE7-β-CD, SBE11-β-CD, and SBE4-γ-CD which correspond to SAE-CD derivatives of the formula I wherein n=5, 5, 5 and 6; m is 4; and there are 4, 7, 11 and 4 sulfoalkyl ether substituents present, respectively. It has been found that these SAE-CD derivatives increase the solubility of poorly water soluble sedative hypnotic drugs, especially etomidate, to varying degrees.

By “therapeutic agent/SAE-CD complex” is generally meant a clathrate or inclusion complex of a sulfoalkyl ether cyclodextrin derivative of the formula (1) and a therapeutic agent. By “complexed” is meant “being part of a clathrate or inclusion complex with”, i.e., a complexed therapeutic agent is part of a clathrate or inclusion complex with a sulfoalkyl ether cyclodextrin derivative. By “major portion” is meant at least about 50% by weight of the therapeutic compound. Thus, a formulation according to the present invention will contain a therapeutic agent of which more than about 50% by weight is complexed with an SAE-CD. In various embodiments, preferably greater than 60% by weight, more preferably greater than 75% by weight, even more preferably greater than 90% by weight, and most preferably greater than 95% by weight of the therapeutic agent will remain complexed with an SAE-CD while in the pharmaceutical formulation. The actual percent of drug that is complexed will vary according to the complexation equilibrium constant characterizing the complexation of a specific SAE- CD to a specific drug. For example, etomidate has a solubility of about 0.45 mg/mL in 0.025 M phosphate buffer at pH 7.0, and needs only about a 10 mM (21 mg/mL) CAPTISOL® cyclodextrin solution to achieve the 2 mg/mL formulation. In this case the amount complexed is only 75%. Generally, the greater the percentage of drug that is complexed, the more effectively the SAE-CD will reduce pain on injection. It should be understood that the degree to which pain on injection is reduced may vary according to the site of injection. Accordingly, a greater reduction of pain on injection may be expected when the injection site is the arm and a smaller reduction of pain on injection may be expected when the injection site is the back of the hand.

A study (R. Rajewski et al., “The effects of water-soluble etomidate on hemodynamics and sympathetic nerve activity in totally deafferented rabbits”, ASA Meeting Abstracts (2000), October 16, A-610) was conducted to compare parenteral formulations, a first (ETCD) comprising etomidate 2 mg/mL and a sulfoalkyl ether cyclodextrin (5% w/v CAPTISOL® cyclodextrin, CyDex, Inc., Overland Park, Kans.) in 25 mM phosphate buffer, pH 7.4 with 3.4 mg/mL sodium chloride, and a second (ETPG) propylene glycol based formulation (AMIDATE®, Abbott Laboratories, Abbott Park, Ill.) comprising 2 mg/mL etomidate in 35% vol. propylene glycol. The study, as described in Example 1, was conducted to evaluate and compare the effects of the two formulations on hemodynamics and sympathetic outflow. Renal sympathetic nerve activity (RSNA) and mean arterial pressure (MAP) were measured and compared for the formulations. The MAP decreased in both groups of rabbits to the same extent: 70.7±4.6% for ETCD and 68.2±2.6% for ETPG. However, the MAP decreased less steeply for the ETCD formulation than for the ETPG formulation: 1.4±0.2%/sec for ETCD and 2.9±0.8%/sec for ETPG. The RSNA decreased significantly for both groups of rabbits but to different extents: 81.7±15.1% for ETCD and 57.4±13.0% for ETPG. Accordingly, equivalent-weight doses of the formulations (0.6 mg etomidate/kg of body weight) are found to be equipotent in terms of the overall decrease in MAP; however, the ETCD formulation is safer for use as an i.v. induction agent in hemodynamically unstable subjects due to the slower rate of decrease in MAP that it provides.

Another study according to Example 2 was conducted on beagles to determine the pharmacodynamic, pharmacokinetic, and hemodynamic properties of two different etomidate-containing formulations: ETCD2 (according to the invention) and ETPG (AMIDATE®). The pharmacokinetic data obtained is summarized in the table below.

Pharmacokinetic Parameters:

| Parameter | ETPG | ETCD2 |
|---|---|---|
| AUC (ng/ml/min) | 24281 ± 2031 | 20399 ± 3386 |
| Vd (L/kg) | 2.73 ± 0.19 | 2.99 ± 0.50 |
| C1 (L/min/kg) | 0.039 ± 0.004 | 0.048 ± 0.008 |
| T_{1/2} (min) | 48.8 ± 5.4 | 43.8 ± 5.9 |

Accordingly, the formulation according to the present invention is substantially bioequivalent to and exhibits substantially the same pharmacokinetics as the propylene glycol based formulation.

Hemolytic assays are generally used in the field of parenteral formulations to predict whether or not a particular formulation is likely to be unsuitable for injection into the bloodstream of a subject. If the formulation being tested induces a significant amount of hemolysis, that formulation will generally be considered unsuitable for administration to a subject. As detailed above in reference to FIG. 1, the safety of cyclodextrins is often compared by way of in-vitro hemolysis studies. Unlike the other cyclodextrin derivatives, SAE-CD derivatives, in particular those such as CAPTISOL® cyclodextrin (degree of substitution≈7) and SBE4-β-CD (degree of substitution≈4), show essentially no hemolytic behavior in concentrations typically used to solubilize pharmaceutical formulations and exhibit substantially lower membrane damaging potential than the commercially available hydroxypropyl derivatives of cyclodextrin. The extent of hemolysis caused by the ETPG and ETCD2 formulations is determined in-vivo in dogs by comparing the level of hemolysis pre-dose and post-dost for each formulation. The hemolysis data obtained is summarized in the table below.

Hemolysis data showing free hemoglobin in the plasma (mg/dL):

| Formulation | Pre-dose | Post-dose | Difference |
|---|---|---|---|
| ETPG | 7.90 ± 6.91 | 71.32 ± 27.20 | 63.42 |
| ETCD2 | 4.25 ± 0.80 | 6.57 ± 2.51 | 2.52 |

All of the SAE-CD compounds of the present invention induce less hemolysis than parent β-cyclodextrin, and all of the preferred SAE-CD compounds, in particular SBE7-β-CD and SBE4-β-CD, induce less hemolysis than HP-β-CD. The formulation of the invention provides a reduced hemolytic potential as compared to or a reduction in the amount of hemolysis caused by the propylene glycol based formulation or another cyclodextrin derivative-containing formulation. Accordingly, the invention also provides a liquid formulation of a sedative hypnotic agent, wherein the formulation has a reduced hemolytic potential as compared to other cyclodextrin-based formulations.

The clinical effect of the ETPG and ETCD2 formulations was compared. The table below provides a summary of the data obtained.

Clinical effect:

| Parameter | ETPG | ETCD2 |
|---|---|---|
| Sleep induction time (sec) | 21 | 22 |
| Sleep time (min) | 9 | 8 |
| Maximal change in heart | −20 | −8 |
| rate (%) | | |
| Respiratory rate after | 62-255 | 59-165 |
| administration (%) | | |

Accordingly, the formulation of the invention is substantially bioequivalent with the AMIDATE® formulation in terms of its overall clinical effect.

The effect of route of administration upon the clinical benefit provided by the formulation of the invention was determined by comparing i.v. and i.m. administration to dogs in Example 2. Unlike other known formulations, the present formulation demonstrated efficacy, albeit inconsistent, during i.m. administration when administered at a dose of 4 mg/ml. No evidence of pain or discomfort on injection was observed in the dogs when administered intramuscularly or intravenously. Accordingly, the formulation of the present invention also provides an etomidate-containing parenteral formulation exhibiting reduced pain on injection as compared to a propylene glycol based formulation. Moreover, the invention provides a method of administering etomidate to induce or maintain sedation comprising the step of intramuscularly administering a liquid formulation comprising an SAE-CD and an effective amount of etomidate.

A different route of administration, subcutaneous (sc), was also compared to the iv route for administration of the solutions of the invention as described in Example 3. The results of the study, depicted in FIG. 4, indicate that the sc route of administration can be used to provide therapeutically effective amounts of etomidate over a more sustained time period relative to the iv administration. Accordingly, the present invention provides a parenteral formulation that can be administered intramuscularly, subcutaneously or intravenously.

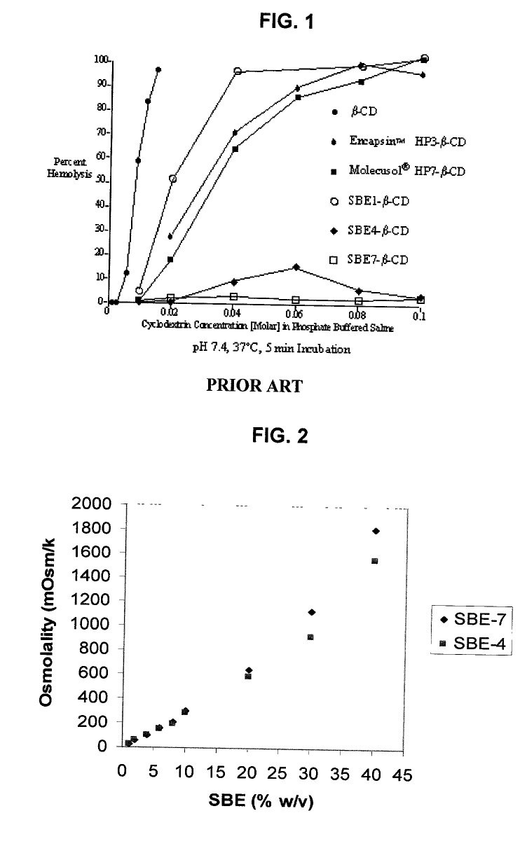

FIG. 2 depicts the relationship between osmolality and concentration of two sulfobutyl ether cyclodextrins in an aqueous solution at room temperature. At concentrations up to about 20% to 30% wt., the relationship is linear. At SBE-CD concentrations suitable for dissolution of etomidate, the solution is slightly hypotonic requiring the addition of a small amount of a tonicity modifying agent to bring the osmolality of about 300 mOsm.

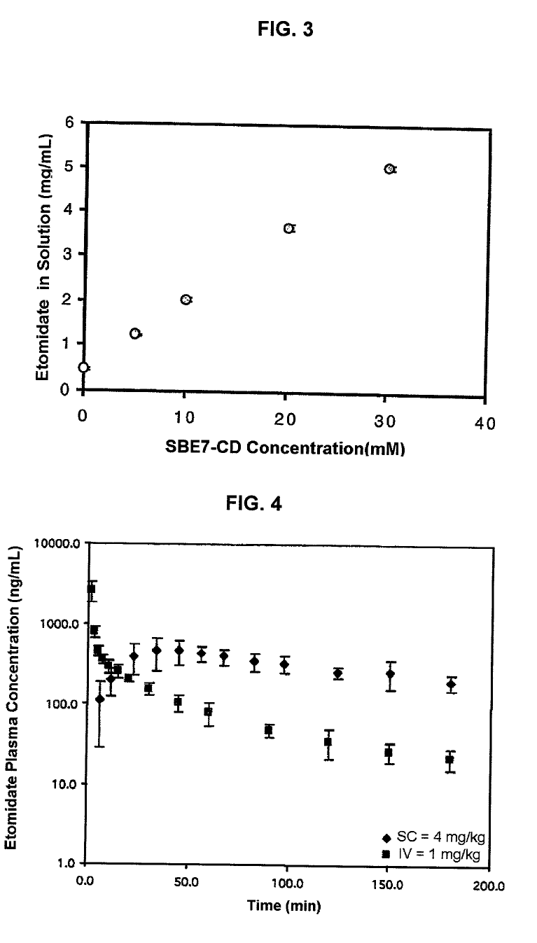

The results of a phase solubility study on the binding of etomidate to an SAE-CD, in particular, SBE7-β-CD are depicted in FIG. 3. The phase solubility study was conducted in a phosphate buffer (25 mM, pH 7.4) at 25° C. Under these conditions, the binding constant was determined to be approximately 445 M⁻¹ as determined by the equilibrium solubility technique (T. Higuchi et al. in “Advances in Analytical Chemistry and Instrumentation Vol. 4”; C. N. Reilly ed.; John Wiley & Sons, Inc, 1965, pp. 117-212).

To raise the aqueous concentration of etomidate from its solubility of 0.45 mg/mL in 25mM phosphate buffer, pH 7.4, to the required formulation concentration of 2.0 mg/mL requires a 5% w/v solution of CAPTISOL® cyclodextrin. Allowing for a 10% deviation in either CAPTISOL® cyclodextrin or etomidate concentration generally requires a CAPTISOL® cyclodextrin concentration of approximately 3% w/v. This concentration will maintain the etomidate in solution under the anticipated extremes of environmental and manufacturing conditions.

At CAPTISOL® cyclodextrin concentrations above about 3% w/v, the equilibrium process of Equations 1 and 2 are shifted to the formation of more complex. At a fixed total concentration of etomidate, this serves to decrease the concentration of free (uncomplexed) etomidate. Thus, the SAE-CD will generally be, but need not be, present in excess of the etomidate.

By knowing the binding constant, one can estimate the amount of SAE-CD required to completely dissolve etomidate and thus determine an optimal molar ratio for the components. However, the ratio of sedative hypnotic agent:SAE-CD present in the formulation will depend on a number of factors, such as, the intrinsic solubility of the agent, the expected dose of the agent, and the binding constant for inclusion complexation between the specific drug (agent) and the specific SAE-CD. These factors combined will determine the amount of SAE-CD needed in the dosage form and therefore the ratio of SAE-CD: therapeutic agent.

The ratio of SAE-CD to etomidate can be varied and still provide a product according to the present invention. A complexation constant of 445 M⁻¹ and a etomidate concentration of 2 mg/mL generally requires a minimum CAPTISOL® cyclodextrin solution concentration of 21.1 mg/mL to keep the etomidate soluble. This corresponds to a mole ratio of about 1.19 or about 1.1 (CAPTISOL® cyclodextrin/etomidate).

However, the amount of each ingredient in a pharmaceutical formulation generally falls within a predetermined range. If a ±10% variation in etomidate concentration is allowed during manufacturing, there might be occasion where the etomidate concentration is 2.2 mg/mL or 0.009 M. Assuming the complexation constant of 445 M⁻¹, then the minimum amount of CAPTISOL® cyclodextrin needed in that formulation is 23.9 mg/mL or 0.011 M. This formulation would have a CAPTISOL® cyclodextrin/etomidate mole ratio of about 1.23. If we set this as the lower limit of the CAPTISOL® cyclodextrin concentration (90% limit, again assuming a potential ±10% variation), then the target CAPTISOL® cyclodextrin concentration would be about 26.6 mg/mL (100%) and the 110% limit would be about 29.2 mg/mL (0.0135 M). On the other hand, if the CAPTISOL® cyclodextrin concentration was on the high side at 0.0135 M and etomidate was on the low side (90% or 1.8 mg/mL) at 0.00737 M, the molar ratio would be about 1.83 or about 1.9 (CAPTISOL® cyclodextrin to etomidate).

If the pH is changed in the formulation and the intrinsic solubility drops to the reported value of 0.0045 mg/100 mL in water, then more CAPTISOL® cyclodextrin would be required to achieve solubility at 2 mg/mL Etomidate. Using the logic above, a CAPTISOL® cyclodextrin to etomidate mole ratio of up to 2.2 might be required. A ratio higher than this might actually lead to less pain on injection by complexing more of the free etomidate.

At a 5.5% wt./v content SAE-CD and 1.8% wt./v of etomidate approximates a molar ratio of 3.5:1 of SAE-CD:etomidate.

Accordingly, the generally minimum effective CAPTISOL® cyclodextrin/etomidate molar ratio is about 1.1, based upon the complexation constants above, and the maximum CAPTISOL® cyclodextrin/etomidate molar ratio is about 2.2, based upon the above-described formulation considerations. It is possible, however, that a higher ratio would be required or desired. Generally, the molar ratio will fall between the range of 1 to 5 CAPTISOL® cyclodextrin/etomidate. A ratio in the range of about 5 to 10 CAPTISOL® cyclodextrin/etomidate is also suitable.

The chemical stability of the formulation contemplated by the present invention has been performed. The table below depicts the stability data of an etomidate formulation in SBE7-β-CD as a function of days. In that study the present formulation comprising 2 mg/mL etomidate in SBE-CD was sterile filtered through a filter of pore size 0.22 microns and stored under a variety of conditions in glass ampoules. The samples were subjected to chromatographic analysis with UV detection.

| | | | Activation | Predicted |
|---|---|---|---|---|
| Temp | Half-life | Rate Constant | Energy | Shelf-Life |
| (° C.) | (Days) | (Days⁻¹) | (kcal mol⁻¹) | (Days) |
| 60 | 123 | 0.0056 | | |
| 70 | 63 | 0.0111 | | |
| 80 | 30 | 0.0230 | | |
| 25 | 2328 | 0.0003 | 17.6 | 354 |

Accordingly, the present invention provides a parenteral formulation having approximately a one-year shelf-life, thereby making the formulation suitable for commercialization.

It should be understood that other SAE-CD compounds of the Formula 1 may be used in the liquid formulation of the invention. These other SAE-CD compounds differ from CAPTISOL® cyclodextrin in their degree of substitution by sulfoalkyl groups, the number of carbons in the sulfoalkyl chain, their molecular weight, the size of the base cyclodextrin used to form the SAE-CD and or their substitution patterns. In addition, the derivatization of β-cyclodextrin with sulfoalkyl groups occurs in a controlled, although not exact manner. For this reason, the degree of substitution is actually a number representing the average number of sulfoalkyl groups per cyclodextrin molecule. In addition, the regiochemistry of substitution of the hydroxyl groups of the cyclodextrin is variable with regard to the substitution of specific hydroxyl groups of the hexose ring. For this reason, sulfoalkyl substitution of the different hydroxyl groups is likely to occur during manufacture of the SAE-CD, and a particular SAE-CD will possess a preferential, although not exclusive or specific, substitution pattern. Given the above, the molecular weight of a particular SAE-CD may vary from batch to batch and will vary from SAE-CD to SAE-CD. All of these variations can lead to changes in the complexation equilibrium constant K_{1:1} which in turn will affect the required ratio of SAE-CD to etomidate. The complexation constant is also somewhat variable with temperature and allowances in the ratio are required such that the agent remains solubilized during the temperature fluctuations that can occur during manufacture, storage, transport and use. Accordingly, the ratio of SAE-CD/etomidate may vary within the range of about 1 to 10, or about 1.1 to 5.

The liquid formulation of the invention may also be converted to a solid formulation for reconstitution. A reconstitutable solid pharmaceutical composition according to the invention comprises a sedative hypnotic agent, an SAE-CD and optionally at least one other pharmaceutical excipient. This composition is reconstituted with an aqueous liquid to form a liquid formulation that is administered by injection or infusion to a subject. The composition can comprise an admixture of a solid SAE-CD and a sedative hypnotic agent-containing solid comprising a sedative hypnotic agent and at least one solid pharmaceutical excipient, such that a major portion of the sedative hypnotic agent is not complexed with the SAE-CD prior to reconstitution. Alternatively, the composition can comprise a solid mixture of an SAE-CD and a sedative hypnotic agent, wherein a major portion of the sedative hypnotic is complexed with the SAE-CD prior to reconstitution.

The reconstitutable formulation is prepared according to any of the following processes. A liquid formulation of the invention is first prepared, then a solid is formed by lyophilization (freeze-drying), spray-drying, spray freeze-drying, antisolvent precipitation, various processes utilizing supercritical or near supercritical fluids, or other methods known to those of ordinary skill in the art to make a powder.

The liquid formulation used in the preparation of the solid formulation, may be prepared as described for the liquid formulation of the invention. It may also be prepared to contain an SAE-CD and the sedative hypnotic agent at concentrations greater than typically used in the liquid formulation of the invention, while maintaining the same SAE-CD:sedative hypnotic agent ratio. The greater concentrations can facilitate several of the processes for isolation of the solid formulation.

The invention also provides a pharmaceutical kit comprising a first container containing a liquid vehicle and a second container containing a reconstitutable solid pharmaceutical composition as described above. The liquid vehicle comprises an aqueous liquid carrier such as water, aqueous alcohol, or aqueous organic solvent.

A complexation-enhancing agent is a compound, or compounds, that enhance(s) the complexation of the etomidate with the SAE-CD. Suitable complexation enhancing agents include one or more pharmacologically inert water soluble polymers, hydroxy acids and other organic compounds typically used in liquid formulations to enhance the complexation of a particular agent with cyclodextrins. When a complexation-enhancing agent is present, the ratio of CAPTISOL® cyclodextrin/etomidate can be changed. Generally, the ratio of CAPTISOL® cyclodextrin/etomidate will fall within the range of about 0.5 to 2.5.

Suitable water soluble polymers include water soluble natural polymers, water soluble semisynthetic polymers (such as the water soluble derivatives of cellulose) and water soluble synthetic polymers. The natural polymers include polysaccharides such as inulin, pectins, algin derivatives and agar, and polypeptides such as casein and gelatin. The semi-synthetic polymers include cellulose derivatives such as methylcellulose, hydroxyethyl cellulose, hydroxypropyl cellulose, their mixed ethers such as hydroxypropyl methylcellulose and other mixed ethers such as hydroxyethyl ethylcellulose and hydroxypropyl ethylcellulose, hydroxypropyl methylcellulose phthalate and carboxymethylcellulose and its salts, especially sodium carboxymethylcellulose. The synthetic polymers include polyoxyethylene derivatives (polyethylene glycols) and polyvinyl derivatives (polyvinyl alcohol, polyvinylpyrrolidone and polystyrene sulfonate) and various copolymers of acrylic acid (e.g. carbomer).

Suitable hydroxy acids include by way of example, and without limitation, citric acid, malic acid, lactic acid, and tartaric acid and others known to those of ordinary skill in the art.

A solubility-enhancing agent is a compound, or compounds, that enhance(s) the solubility of the etomidate in the liquid formulation. A solubility-enhancing agent can be added to the aqueous liquid formulation of the invention. When the solubility-enhancing agent is present, the ratio of CAPTISOL® cyclodextrin/etomidate can be changed. Generally, the ratio of CAPTISOL® cyclodextrin/etomidate will fall within the range of about 0.5 to 2.5.

Suitable solubility enhancing agents include one or more organic solvents, detergents, soaps, surfactant and other organic compounds typically used in parenteral formulations to enhance the solubility of a particular agent.

Suitable organic solvents include, for example, ethanol, glycerin, polyethylene glycols, propylene glycol, poloxomers, and others known to those of ordinary skill in the art.

The etomidate is generally present in amounts ranging from about 0.1 to 0.5% w/v. Dosage levels of etomidate for producing general anesthesia, both induction (for example about 0.2 and 0.6 mg/kg of body weight for an adult) and maintenance, and for producing a sedative effect, may be derived from the substantial literature on etomidate. Furthermore, the anesthetist and/or physician would modify the dose to achieve the desired effect in a patient in accordance with the conventional skill in the art. Smaller increments of intravenous etomidate may be administered to adult patients during short operative procedures to supplement subpotent anesthetic agents, such as nitrous oxide. The dosage employed under these conditions, although usually smaller than the original induction dose, must be individualized.

Soaps and synthetic detergents may be employed as surfactants and as vehicles for detergent compositions. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl β-aminopropionates and 2-alkylimidazoline quaternary ammonium salts; synthetic or naturally occurring phosphatide; others known to those of ordinary skill in the art; and mixtures thereof.

Suitable soaps include fatty acid alkali metal, ammonium, triethanolamine salts and others known to those of ordinary skill in the art.

The dosage form of the invention can also include oils, for example, fixed oils, such as peanut oil, sesame oil, cottonseed oil, corn oil and olive oil; fatty acids, such as oleic acid, stearic acid and isostearic acid; and fatty acid esters, such as ethyl oleate, isopropyl myristate, fatty acid glycerides and acetylated fatty acid glycerides. It can also include alcohols, such as ethanol, isopropanol, hexadecyl alcohol, glycerol and propylene glycol; glycerol ketals, such as 2,2-dimethyl-1,3-dioxolane-4-methanol; ethers, such as poly(ethyleneglycol) 450; with petroleum hydrocarbons, such as mineral oil and petrolatum; water; or with mixtures thereof; with or without the addition of a pharmaceutically suitable surfactant, suspending agent or emulsifying agent.

It should be understood, that compounds used in the art of pharmaceutical formulations generally serve a variety of functions or purposes. Thus, if a compound named herein is mentioned only once or is used to define more than one term herein, its purpose or function should not be construed as being limited solely to that named purpose(s) or function(s).

Although not necessary, the formulation of the present invention can include a preservative, antioxidant, buffering agent, acidifying agent, alkalizing agent, antibacterial agent, antifungal agent, solubility enhancing agent, complexation enhancing agent, solvent, electrolyte, salt, water, stabilizer, tonicity modifier, antifoaming agent, oil, emulsifying agent, bulking agent, cryoprotectant or a combination thereof.

As used herein, the term “alkalizing agent” is intended to mean a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids and trolamine and others known to those of ordinary skill in the art.

As used herein, the term “acidifying agent” is intended to mean a compound used to provide an acidic medium for product stability. Such compounds include, by way of example and without limitation, acetic acid, acidic amino acids, citric acid, fumaric acid and other alpha hydroxy acids, hydrochloric acid, ascorbic acid, phosphoric acid, sulfuric acid, tartaric acid and nitric acid and others known to those of ordinary skill in the art.

As used herein, the term “preservative” is intended to mean a compound used to prevent the growth of microorganisms. Such compounds include, by way of example and without limitation, benzalkonium chloride, benzethonium chloride, benzoic acid, benzyl alcohol, cetylpyridinium chloride, chlorobutanol, phenol, phenylethyl alcohol, phenylmercuric nitrate, phenylmercuric acetate, thimerosal, metacresol, myristylgamma picolinium chloride, potassium benzoate, potassium sorbate, sodium benzoate, sodium propionate, sorbic acid, thymol, and methyl, ethyl, propyl or butyl parabens and others known to those of ordinary skill in the art. Particularly useful preservatives include EDTA, pentetate, and combinations thereof.

As used herein, the term “antioxidant” is intended to mean an agent that inhibits oxidation and thus is used to prevent the deterioration of preparations by the oxidative process. Such compounds include, by way of example and without limitation, acetone sodium bisulfite, ascorbic acid, ascorbyl palmitate, citric acid, butylated hydroxyanisole, butylated hydroxytoluene, hypophophorous acid, monothioglycerol, propyl gallate, sodium ascorbate, sodium citrate, sodium sulfide, sodium sulfite, sodium bisulfite, sodium formaldehyde sulfoxylate, thioglycolic acid and sodium metabisulfite and others known to those of ordinary skill in the art.

As used herein, the term “buffering agent” is intended to mean a compound used to resist change in pH upon dilution or addition of acid or alkali. Such compounds include, by way of example and without limitation, acetic acid, sodium acetate, adipic acid, benzoic acid, sodium benzoate, citric acid, maleic acid, monobasic sodium phosphate, dibasic sodium phosphate, lactic acid, tartaric acid, potassium metaphosphate, potassium phosphate, monobasic sodium acetate, sodium bicarbonate, sodium tartrate and sodium citrate anhydrous and dihydrate and others known to those of ordinary skill in the art.

As used herein, the term “stabilizer” is intended to mean a compound used to stabilize the therapeutic agent against physical, chemical, or biochemical process which would reduce the therapeutic activity of the agent. Suitable stabilizers include, by way of example and without limitation, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and other known to those of ordinary skill in the art.

As used herein, the term “tonicity modifier” is intended to mean a compound or compounds that can be used to adjust the tonicity of the liquid formulation. Suitable tonicity modifiers include glycerin, lactose, mannitol, dextrose, sodium chloride, sodium sulfate, sorbitol, trehalose and others known to those of ordinary skill in the art. In one embodiment, the tonicity of the liquid formulation approximates the tonicity of blood or plasma.

As used herein, the term “antifoaming agent” is intended to mean a compound or compounds that prevents or reduces the amount of foaming that forms on the surface of the liquid formulation. Suitable antifoaming agents include dimethicone, simethicone, octoxynol and others known to those of ordinary skill in the art.

As used herein, the term “bulking agent” is intended to mean a compound used to add bulk to the lyophilized product and/or assist in the control of the properties of the formulation during lyophilization. Such compounds include, by way of example and without limitation, dextran, trehalose, sucrose, polyvinylpyrrolidone, lactose, inositol, sorbitol, dimethylsulfoxide, glycerol, albumin, calcium lactobionate, and others known to those of ordinary skill in the art.

As used herein, the term “cryoprotectant” is intended to mean a compound used to protect an active therapeutic agent from physical or chemical degradation during lyophilization. Such compounds include, by way of example and without limitation, dimethyl sulfoxide, glycerol, trehalose, propylene glycol, polyethylene glycol, and others known to those of ordinary skill in the art.

As used herein, the term “emulsifier” or “emulsifying agent” is intended to mean a compound added to one or more of the phase components of an emulsion for the purpose of stabilizing the droplets of the internal phase within the external phase. Such compounds include, by way of example and without limitation, lecithin, polyoxylethylene-polyoxypropylene ethers, polyoxylethylene-sorbitan monolaurate, polysorbates, sorbitan esters, stearyl alcohol, tyloxapol, tragacanth, xanthan gum, acacia, agar, alginic acid, sodium alginate, bentonite, carbomer, carboxymethyl cellulose sodium, cholesterol, gelatin, hydroxyethyl cellulose, hydroxypropyl cellulose, octoxynol, oleyl alcohol, polyvinyl alcohol, povidone, propylene glycol monostearate, sodium lauryl sulfate, and others known to those of ordinary skill in the art.

The formulation of the invention can also include water, organic solvent(s) and combinations thereof. In particular embodiments, the formulation includes alcohol, water, and saline.

The formulation of the invention can also include biological salt(s), sodium chloride, potassium chloride, or other electrolyte(s).

The chemical stability of the liquid formulation varies with pH, thus proper selection of pH is necessary for a stable preparation. The chemical stability of the liquid formulation can also be enhanced by converting the liquid formulation to a solid formulation.

The liquid formulation can include a buffering agent, acidifying agent, alkalizing agent or combination thereof as a means of controlling the pH of the liquid. The pH of the liquid formulation will generally range from about 6.0-8.0. In one embodiment, the pH of the liquid formulation approximates the pH of blood or plasma. Exemplary buffering agents, acidifying agents and alkalizing agents are disclosed herein. In one embodiment, the buffering agent is a phosphate buffer present at a concentration of about 0.025 M of the liquid formulation, with a pH of 7.4.

Etomidate is subject to light catalyzed degradation. Therefore, the liquid formulation is generally stored in a light-resistant or lightproof container. Suitable containers, such as vials, bottles, syringes or ampoules, can be made of amber-colored glass, light-blocking plastic, or paper, plastic, foil, metal or otherwise covered glass and/or plastic. The combined use of an antioxidant, light-resistant or lightproof container and an oxygen-free or oxygen-reduced environment provides the greatest protection against degradation of the etomidate. Depending upon the level of exposure to light, the light catalyzed degradation of etomidate might be reduced by addition of an SAE-CD.

The liquid formulation of the invention can be provided in an ampoule, syringe, bottle, vial or other such container typically used for parenteral formulations.

Etomidate might adsorb onto plastic or glass surfaces. The adsorption might be minimized by including an SAE-CD in a liquid formulation containing etomidate. Accordingly, the invention also provides a method of reducing the adsorption of etomidate onto a plastic or glass surface comprising the step of including an SAE-CD and etomidate in a liquid formulation in a plastic or glass container.

Other therapeutic agents such as local anesthetics can be included in the formulation of the invention. When present, these other therapeutic agents may or may not bind or complex with the SAE-CD. Representative local anesthetics include benzocaine, procaine, lidocaine, piperocaine, tetracaine, lignocaine, prolicaine, bupivacaine, proxymetacaine, ropivacaine, and dibucaine.

The liquid formulation of the invention can be prepared by numerous different methods. According to one method, a first aqueous solution comprising SAE-CD is prepared. Then, a second solution comprising a sedative hypnotic is prepared. Finally, the first and second solutions are mixed to form the liquid formulation. The first and second solutions can independently comprise other excipients and agents described herein. Additionally, the second solution can be water and/or organic solvent-based.

Another method of preparation is similar to the above-described method except that the sedative hypnotic is added directly to the first solution without formation of the second solution.

A third method of preparing the liquid formulation is similar to the above-described first method except that the SAE-CD is added directly to an aqueous second solution containing the sedative hypnotic without formation of the first solution.

A fourth method of preparing the liquid formulation comprises the steps of adding an aqueous solution comprising a sedative hypnotic to a powdered or particulate SAE-CD and mixing the solution until the SAE-CD has dissolved.

The liquid formulation of the invention can be provided in a kit. The kit will comprise a first pharmaceutical composition comprising an SAE-CD and a second pharmaceutical composition comprising a sedative hypnotic agent. The first and second formulations can be mixed and formulated as a liquid dosage form prior to administration to a subject. Either one or both of the first and second pharmaceutical compositions can comprise additional pharmaceutical excipients. The kit is available in various forms.

In a first kit, the first and second pharmaceutical compositions are provided in separate containers or in separate chambers of a container having two or more chambers. The first and second pharmaceutical compositions may be independently provided in solid or liquid form. For example, the SAE-CD can be provided in a solution form and the sedative hypnotic agent can be provided in solid form. According to one embodiment the kit would further comprise a pharmaceutically acceptable liquid carrier used to suspend and dissolve the first and/or second pharmaceutical compositions. Alternatively, a liquid carrier is independently included with the first and/or second pharmaceutical composition. The liquid carrier, however, can also be provided in a container or chamber separate from the first and second pharmaceutical compositions. As above, the first pharmaceutical composition, the second pharmaceutical composition and the liquid carrier can independently comprise a preservative, an antioxidant, a buffering agent, an acidifying agent, saline, an electrolyte, another therapeutic agent, an alkalizing agent, an antimicrobial agent, an antifungal agent, solubility enhancing agent or a combination thereof.

The liquid formulation of the invention can be provided as a dosage form including a prefilled vial, prefilled bottle, prefilled syringe, prefilled ampoule or plural ones thereof. Generally, a prefilled container will contain at least a unit dosage form of the sedative hypnotic agent.

The term “unit dosage form” is used herein to mean a single or multiple dose form containing a quantity of the active ingredient and the diluent or carrier, said quantity being such that one or more predetermined units are normally required for a single therapeutic administration. In the case of multiple dose forms, such as liquid-filled ampoules, said predetermined unit will be one fraction such as a half or quarter of the multiple dose form. It will be understood that the specific dose level for any patient will depend upon a variety of factors including the indication being treated, the therapeutic agent employed, the activity of the therapeutic agent, severity of the indication, patient health, age, sex, weight, diet, and pharmacological response, the specific dosage form employed and other such factors.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “patient” is taken to mean warm blooded animals such as mammals, for example, cats, dogs, mice, guinea pigs, horses, bovine cows, sheep and humans.

The liquid formulation of the invention will comprise an effective amount of etomidate. By the term “effective amount”, it is understood that a therapeutically effective amount is contemplated. A therapeutically effective amount is the amount or quantity of etomidate that is sufficient to elicit the required or desired therapeutic response, or in other words, the amount that is sufficient to elicit an appreciable biological response when administered to a subject.

The present formulation containing SAE-CD and a sedative hypnotic agent can be sterile filtered through filters having pores sizes of 0.1 microns or larger, or pore sizes of about 0.1 microns, 0.2 microns, 0.22 microns, 0.3 microns, 0.45 microns or larger. Accordingly, a method of preparing a sterile SAE-CD/sedative hypnotic formulation can comprise the step of sterile filtering the formulation through a filtration medium having a pore size of 0.45 microns or smaller.

As with other sedative hypnotic-containing formulations, the present formulation is used to induce hypnosis, induce sedation and/or maintain sedation in a subject. Hypnosis and sedation are induced by administering to a subject a sufficient amount of the liquid formulation of the invention, by injection or infusion, over a sufficient period of time to induce hypnosis and/or sedation in the subject. Sedation is maintained by administering a sufficient amount of the liquid formulation of the invention, by periodic injection or continuous infusion. In general, induction of hypnosis or sedation can be performed by rapid or slow administration of the sedative hypnotic depending upon the needs of a particular subject. Maintenance of sedation is typically conducted by administering a lower dose of sedative hypnotic to an already sedated subject. A subject may be previously sedated with another drug and then administered the sedative hypnotic according to the invention. Likewise, sedation or hypnosis can be induced in a subject with another drug and subsequently maintained by administration of a sedative hypnotic according to the invention. Other injectable agents used alone or in combination with the sedative hypnotic of the invention include the benzodiazepines such as midazolam, and flunitrazepam; narcotics such as morphine, buprenorphine, fentanyl, alfentanyl, sufentanyl, and remifentanyl; barbiturates such as thiopentone and methohexital; and other agents such as propofol, ketamine, thiopentone, and alphaxalone/alphadolone.

In view of the above description and the examples below, one of ordinary skill in the art will be able to practice the invention as claimed without undue experimentation. The foregoing will be better understood with reference to the following examples that detail certain procedures for the preparation of formulations according to the present invention. All references made to these examples are for the purposes of illustration. The following examples should not be considered exhaustive, but merely illustrative of only a few of the many embodiments contemplated by the present invention.

### EXAMPLE 1

The following procedure was used to conduct a study of the effects of etomidate on the hemodynamics and sympathetic nerve activity of totally deafferented rabbits.

The following formulations were used:

ETCD:

| | |
|---|---|
| Etomidate | 2.0 mg/ml |
| SBE7-β-CD | 5% wt./v˜23 mM) |
| NaCl | 3.4 mg/ml |
| 25 mM Phosphate Buffer | pH 7.4 |
| Osmolality | 300 mOsm |

ETPG (AMIDATE ®):

| | |
|---|---|
| Etomidate | 2.0 mg/ml |
| Propylene glycol | 35% vol. |
| Water | to volume |
| pH | ˜5 |
| Osmolality | >4500 mOsm |

The exemplary formulation of the invention was made by dissolving the cyclodextrin in the buffer solution also containing salt. The etomidate was added to the SAE-CD containing solution until a concentration of about 2.0 mg/ml of etomidate was reached. The formulation had an osmolality of 300 mOsm.

Under basal anesthesia and controlled ventilation, the animals underwent surgical preparation (isolation of the left renal sympathetic nerve, combined denervation of bilateral carotid sinus, aortic and vagal nerves). After hemodynamic stabilization, the deafferented rabbits were divided into two groups: one group receiving ETCD and the other group receiving ETPG. The animals received bolus i.v. injections of either ETCD or ETPG over a period of 5 seconds such that the total amount of etomidate administered was 0.6 mg/kg of body weight. RSNA and MAP along with EKG were continuously recorded for 30 min. Repeated measure ANOVA followed by Newman-Keuls procedure was used for statistical analysis. P<0.05 was considered significant.

### EXAMPLE 2

The following procedure was used to conduct a study of the pharmacodynamic, pharmacokinetic, and hemodynamic properties in 4 dogs of three different etomidate-containing formulations.

The following formulations were used:

ETCD2:

| | |
|---|---|
| Etomidate | 2 mg/ml |
| SBE7-β-CD | 5% w/v (˜23 mM) Phosphate |
| buffer (25 mM, pH 7.4) | to volume |
| NaCl | 3.4 mg/ml |
| Osmolality | ˜300 mOsm |

ETCD3:

| | |
|---|---|
| Etomidate | 4 mg/ml |
| SBE7-β-CD | 5% w/v (˜23 mM) Phosphate |
| buffer (25 mM, pH 7.4) | to volume |

ETPG (AMIDATE ®):

| | |
|---|---|
| Etomidate | 2.0 mg/ml |
| Propylene glycol | 35% vol. |
| pH | ˜5 |
| Osmolality | >4500 mOsm |

The exemplary formulation of the invention was made by dissolving the cyclodextrin in the buffer solution also containing salt. The etomidate was added to the SAE-CD containing solution until a concentration of about 2 mg/ml (ETCD2) or 4 mg/mL (ETCD3) of etomidate was reached.

Four male beagles weighing 10.6 to 14.1 kg received i.v. bolus injections of ETCD2 and ETPG and an im injection of ETCD3 in a crossover design with a 1 week washout between doses. The iv doses were administered over a period of 15 sec such that the total amount of etomidate administered was about 1 mg/kg of body weight. The im doses were administered at a higher concentration (4 mg/mL etomidate) and higher dose (2 mg/kg), divided into two lumbar sites. The study was conducted on four dogs, each receiving three formulations with one week between each dose. Blood samples were drawn periodically over a period of three hours total starting from shortly before injecting dogs with the etomidate containing solution. The pharmacokinetic data was obtained by analysis of the raw data using the SAAM II application in a 2 compartment model. The AUC, CL, Vdist, Kₑₗ and t_{½} were calculated. The presence of free hemoglobin in plasma, indicating hemolysis, was determined by comparing pre- and post dose samples of whole blood in dogs administered the ETCD formulation of the present invention. A SIGMA diagnostic kit (527-A) and a spectrophotometric analysis at 600 nm was used to quantify hemolysis. The EEG analysis included a power spectrum analysis. The sleep induction time (SIT), sleep time (ST), maximal change in heart rate, (ΔHRmax) and respiratory rate (RR) were measured

### EXAMPLE 3

The following procedure was used to conduct a study of the pharmacokinetic properties in 4 dogs of two different etomidate-containing formulations.

The following formulations were used:

ETCD2:

| | |
|---|---|
| Etomidate | 2 mg/ml |
| SBE7-β-CD | 5% w/v (˜23 mM) Phosphate |
| buffer (25 mM, pH 7.4) | to volume |
| NaCl | 3.4 mg/ml |
| Osmolality | ˜300 mOsm |

ETCD4:

| | |
|---|---|
| Etomidate | 12 mg/mL |
| SBE7-β-CD | 20% w/v (˜92 mM) |
| Phosphate buffer (25 mM, pH 7.4) | to volume |

Four male beagles weighing 10.6 to 14.1 kg received an iv bolus injection of ETCD2 and a subcutaneous (sc) injection of ETCD4 in a crossover design with a 1 week washout between doses. The iv doses were administered over a period of 15 sec such that the total amount of etomidate administered was about 1 mg/kg of body weight. The sc doses were administered at a higher concentration (12 mg/mL etomidate) and higher dose (4 mg/kg), divided into two lumbar sites. Blood samples were drawn periodically over a period of three hours total starting from shortly before injecting dogs with the etomidate containing solution.

The clarity of the liquid formulations described herein can be determined visually by comparison to standard solutions of known clarity. The clarity can also be determined by transmittance spectrophotometry at a wavelength of 800 nm. Using either method, the solutions prepared according to the invention were determined to be at least visually clear.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1. A pharmaceutical formulation comprising:
etomidate;
a sulfoalkyl ether cyclodextrin (SAE-CD); and
a liquid carrier, wherein the molar ratio of SAE-CD to etomidate is in the range of about 1.1:1 to 10:1.

2. The formulation of claim 1, wherein the molar ratio of SAE-CD to etomidate is in the range of about 1.1:1 to about 3.5:1.

3. The formulation of claim 2, wherein the etomidate is present in an amount of about 0.5 to 40 mg/mL.

4. The formulation of claim 3, wherein the SAE-CD is present in an amount of about 0.0002 to 0.25 M.

5. The formulation of claim 1, wherein the SAE-CD is a compound of the Formula 1 or a combination thereof wherein:
n is 4, 5 or 6;
R₁, R₂, R₃, R₄, R₅, R₆, R₇, R₈ and R₉ are each, independently, —O— or a-O—(C2-C6 alkylene)-SO₃⁻ group, wherein at least one of R₁ and R₂ is independently a —O—(C2-C6 alkylene)-SO₃⁻ group; and
S₁, S₂, S₃, S₄, S₅, S₆, S₇, S₈ and S₉ are each a pharmaceutically acceptable cation.

6. The formulation of claim 5, wherein the compound of Formula 1 is SBE-7-β-CD or SBE-4-β-CD.

7. The formulation of claim 5, wherein the molar ratio of SAE-CD to etomidate is in the range of about 1.1:1 to about 3.5:1.

8. The formulation of claim 7, wherein the etomidate is present in an amount of about 0.5 to 40 mg/mL.

9. The formulation of claim 8, wherein the SAE-CD is present in an amount of about 0.0002 to 0.25M.

10. The formulation of claims 1-8 or 9 further comprising a preservative, antioxidant, buffering agent, acidifying agent, alkalizing agent, antibacterial agent, another therapeutic agent, antifungal agent, solubility enhancing agent, complexation enhancing agent, organic solvent, electrolyte, salt, stabilizer, tonicity modifier, antifoaming agent or a combination thereof.

11. The formulation of claim 10, wherein the formulation comprises less than about 5 ppm of oxygen gas.

12. The formulation of claim 10, wherein the other therapeutic agent is a local anesthetic agent.

13. The formulation of claim 12, wherein the anesthetic agent is selected from the group consisting of benzocaine, procaine, lidocaine, piperocaine, tetracaine, lignocaine, prolicaine, bupivacaine, proxymetacaine, ropivacaine, dibucaine and a combination thereof.

14. The formulation of claim 1, wherein the liquid formulation has been sterile filtered through a filtration medium having a pore size of about 0.22 microns or smaller.

15. The formulation of claims 1-9 or 14, wherein the formulation exhibits improved chemical stability as compared to a formulation containing propylene glycol.

16. A liquid formulation of etomidate that under storage does not undergo transesterification with a component of the liquid formulation.

17. A pharmaceutical kit comprising:
a first pharmaceutical composition comprising an SAE-CD; and
a second pharmaceutical composition comprising etomidate;
wherein at least the first and second pharmaceutical compositions can be mixed with a liquid carrier to form a liquid dosage form prior to administration to a subject.

18. The pharmaceutical kit of claim 17, wherein the first and second composition independently further comprise one or more pharmaceutical excipients.

19. The pharmaceutical kit of claim 17, wherein the first and second pharmaceutical compositions are provided in separate containers or in separate chambers of a container having two or more chambers.

20. The pharmaceutical kit of claim 17, wherein the kit further comprises a pharmaceutically acceptable liquid carrier used to form the liquid formulation.

21. The pharmaceutical kit of claim 20, wherein the liquid carrier is independently included with the first and/or second pharmaceutical composition.

22. The pharmaceutical kit of claim 20, wherein the liquid carrier is provided in a container or chamber separate from the first and second pharmaceutical compositions.

23. The pharmaceutical kit of claim 20, wherein the first pharmaceutical composition, the second pharmaceutical composition, the liquid carrier or a combination thereof further comprises a preservative, an antioxidant, a buffering agent, an acidifying agent, saline, an electrolyte, another therapeutic agent, an alkalizing agent, an antimicrobial agent, an antifungal agent, a solubility enhancing agent, an emulsifying agent, oil, complexation enhancing agent or a combination thereof.

24. The pharmaceutical kit of claim 23, wherein the other therapeutic agent is a local anesthetic.

25. The pharmaceutical kit of claim 17, wherein the first and/or second pharmaceutical composition further comprises a preservative, an antioxidant, a buffering agent, an acidifying agent, saline, an electrolyte, another therapeutic agent, an alkalizing agent, an antimicrobial agent, an antifungal agent, a solubility enhancing agent, an emulsifying agent, oil, complexation enhancing agent, or a combination thereof.

26. The pharmaceutical kit of claim 25, wherein the other therapeutic agent is a local anesthetic.

27. The pharmaceutical kit of claim 17, wherein the kit is provided chilled.

28. A method of reducing the pain on injection associated with administration of etomidate by injection or intravenous infusion, the method comprising the step of:
including in the liquid formulation a sulfoalkyl ether cyclodextrin and etomidate, wherein the ratio of sulfoalkyl ether cyclodextrin to etomidate is sufficient to provide reduced pain on injection as compared to a propylene glycol based liquid formulation comprising a comparable amount of etomidate.

29. A method of administering etomidate to a subject comprising the step of:
administering to a subject by injection or intravenous infusion a liquid formulation comprising a sulfoalkyl ether cyclodextrin and etomidate, wherein the molar ratio of SAE-CD to etomidate is in the range of about 1.1:1 to 10:1, and etomidate is present in an amount of about 0.5 to 40 mg/ml.

30. The method of claim 29, wherein the SAE-CD is present in an amount of about 0.0002 to 0.25 M.

31. The method of claim 29, wherein the amount of etomidate administered is sufficient to induce hypnosis or sedation in the subject.

32. The method of claim 29, wherein the amount of etomidate administered is sufficient to maintain sedation in a subject in which sedation has already been induced.

33. The method of claims 29-31 or 32, wherein the liquid formulation causes less or no bradycardia in a subject being administered the liquid formulation as compared to a propylene glycol based formulation comprising 35% v/v propylene glycol in water and a comparable amount of etomidate.

34. The method of claims 29-31 or 32, wherein the liquid formulation provides a hemodynamic response similar to that of a propylene glycol based formulation comprising 35% wt. propylene glycol in water and a comparable amount of etomidate.

35. A method of administering etomidate to a subject comprising the step of:
administering to a subject by injection, intravenous infusion, or orally a liquid formulation according to claims 1-8 or 9.

36. A method of administering etomidate to a subject comprising the step of:
administering to a subject by injection, intravenous infusion, or orally a liquid formulation according to claim 10.

37. A method of administering etomidate to a subject comprising the step of:
administering to a subject by injection, intravenous infusion or orally a liquid formulation according to claim 12.

38. A method of administering etomidate to a subject comprising the step of:
administering to a subject by injection, intravenous infusion or orally a liquid formulation according to claim 13.

39. The formulation of claim 8, wherein the SAE-CD is present in an amount of about 0.04 to 50% weight/volume.

40. A reconstitutable solid pharmaceutical composition comprising:
etomidate and a sulfoalkyl ether cyclodextrin (SAE-CD), wherein the molar ratio of SAE-CD to etomidate is in the range of about 1.1:1 to 10:1.

41. The composition of claim 40, wherein the SAE-CD is a compound of the Formula 1 or a combination thereof wherein:
n is 4, 5 or 6;
R₁, R₂, R₃, R₄, R₅, R₆, R₇, R₈ and R₉ are each, independently, —O— or a-O—(C2-C6 alkylene)-SO₃⁻ group, wherein at least one of R₁ and R₂ is independently a —O—(C2-C6 alkylene)-SO₃⁻ group; and
S₁, S₂, S₃, S₄, S₅, S₆, S₇, S₈ and S₉ are each a pharmaceutically acceptable cation.

42. The composition of claim 41, wherein the composition comprises an admixture of a solid SAE-CD, etomidate and optionally at least one solid pharmaceutical excipient, such that a major portion of the etomidate is not complexed with the SAE-CD prior to reconstitution.

43. The composition of claim 41, wherein the composition comprises a solid mixture of an SAE-CD and etomidate, wherein a major portion of the etomidate is complexed with the SAE-CD prior to reconstitution.

44. The composition of claims 40-42 or 43 further comprising a preservative, an antioxidant, a buffering agent, an acidifying agent, saline, an electrolyte, another therapeutic agent, an alkalizing agent, an antimicrobial agent, an antifungal agent, solubility enhancing agent, emulsifying agent, complexation enhancing agent, antifoaming agent, oil, or a combination thereof.

45. The composition of claim 44, wherein the other therapeutic agent is a local anesthetic agent.

46. The composition of claim 45, wherein the local anesthetic agent is selected from the group consisting of benzocaine, procaine, lidocaine, piperocaine, tetracaine, lignocaine, prolicaine, bupivacaine, proxymetacaine, ropivacaine, and dibucaine.

47. The composition of claim 40-42 or 43, wherein the molar ratio of SAE-CD to etomidate is in the range of about 1.1: 1 to about 3.5: 1.

48. The formulation of claim 47, wherein the etomidate is present in an amount of about 0.5 to 40 mg/mL.

49. The formulation of claim 48, wherein the SAE-CD is present in an amount of about 0.0002 to 0.25 M.

50. The formulation of claim 10, wherein the formulation exhibits improved chemical stability as compared to a formulation containing propylene glycol.

51. The formulation of claim 12, wherein the formulation exhibits improved chemical stability as compared to a formulation containing propylene glycol.

52. The method of claim 28, wherein the molar ratio of SAE-CD to etomidate is in the range of about 1.1:1 to 10:1, and etomidate is present in an amount of about 0.5 to 40 mg/ml.

53. The formulation of claim 52, wherein the SAE-CD is present in an amount of about 0.0002 to 0.25 M.

54. The method of claim 29, wherein the molar ratio of SAE-CD to etomidate is in the range of about 1.1:1 to about 3.5:1.
